(19) United States
(12) Patent Application Publication
(10) Pub. No.: US 2021/0128981 A1
(43) Pub. Date: May 6, 2021

(54) System And Method For Evaluating Amateur Athletes For Collegiate Level Placement

(71) Applicant: College Sports Evaluation, LLC, Fort Collins, CO (US)

(72) Inventors: David King, Fort Collins, CO (US); Kelly Salberg, Fort Collins, CO (US); Alyssa Morrison, Fort Collins, CO (US)

(21) Appl. No.: 17/085,533

(22) Filed: Oct. 30, 2020

Related U.S. Application Data
(60) Provisional application No. 62/928,822, filed on Oct. 31, 2019.

(51) Int. Cl. A63B 24/00; G16H 50/30; G16H 50/70; A63B 71/06

(52) U.S. Cl. CPC A63B 24/0062; G16H 50/30; G06Q 50/205; A63B 71/0616; G16H 50/70

(57) ABSTRACT

The invention includes a system and method for analyzing and evaluating amateur athletes for advising athletes on performance abilities that best match placement of the athlete for participation in intercollegiate athletics. The invention provides a computer implemented solution with automated functionality providing athletes reporting information as to their current performance level matched with an appropriate level of intercollegiate participation, as well as reporting information as to how the athlete may improve their performance level to attain a match for a higher level of competition.

### CROSS REFERENCE TO RELATED APPLICATIONS

This application claims the benefit of and priority to Provisional Patent Application No. 62/928,822, filed Oct. 31, 2019. The entire specification and figures of the above-referenced application are hereby incorporated, in their entirety by reference.

### FIELD OF THE INVENTION

The invention relates to a system and method for analyzing and evaluating amateur athletes for purposes of advising athletes on performance abilities that best match placement of the athlete for participation in intercollegiate athletics, and more particularly, to a system and method that provides a computer implemented solution with automated functionality providing athletes reporting information as to their current performance level matched with an appropriate level of intercollegiate participation, as well as reporting information as to how the athlete may improve their performance level to attain the desired level of intercollegiate participation.

### BACKGROUND OF THE INVENTION

Athletics are in important aspect of many cultures. For amateur athletes, participation in athletics is highly regarded for many reasons. Historically, athletic competitions have been a centerpiece for many cultures in which individual athletes and teams received both a tremendous amount of attention and scrutiny. Regardless of the particular sport, athletes begin their careers at an amateur level, most beginning at an early age.

In the US, participating in athletics at a collegiate level defines the pinnacle of success for the great majority of athletes. A small percentage of high school athletes are qualified to participate at the collegiate level. An even smaller percentage of collegiate athletes are qualified to participate at a professional level.

Some athletes are naturally gifted with abilities that enable them to advance to collegiate and professional levels. The great majority of athletes however, do not have dominant athletic abilities and must rely upon hard work and dedication in order to achieve participation at the collegiate level.

As time has progressed, the cost of a college education in the US has skyrocketed. Recent statistics show that a significant percentage of college students incur tremendous financial debt through student loans. The college debt incurred by students is recognized as an endemic problem in the US with no present solutions available for most students.

One avenue to avoid student debt is the ability to obtain a college scholarship. Because of increased participation in amateur athletics coupled with the increasing costs of obtaining a college education, competition among amateur athletes to obtain college scholarships is intense. Parents/guardians of amateur athletes spend a tremendous amount of time and financial resources in supporting their amateur athletes, many of which strive to obtain a college scholarship. For many families, the only possibility to obtain a college education is through an athletic scholarship.

The determination of whether an amateur athlete qualifies for a college athletic scholarship varies greatly among colleges. There are a tremendous number of subjective and objective factors that college coaches evaluate to determine whether an athlete may be a good candidate for receiving a scholarship. However, a traditionally subjective analysis for determining who is a good candidate for a scholarship has progressed towards increased evaluation of objective factors for determining scholarship candidates. This transition to measured objective factors can be attributed to a number of reasons, one of which is the increased number of candidates who apply for scholarships as well as the desire of universities to maximize successful college performers who are scholarship athletes. Football and basketball, for many colleges, provides the great majority of revenue to finance the cost of all of the other sport teams of the university. A successful football or basketball program inevitably results in increased revenue for a university and the ability to better serve all athletes. For these sports, competition for scholarships is particularly intense.

Considering the increased participation of amateur athletes, the costs associated with attending college and a general increase in societal interest in athletics, there is now an industry dedicated to increasing athletic performance for all ages of youth athletes. Similarly, there is also a burgeoning industry associated with increasing athletic performances for collegiate as well as professional athletes.

In the context of evaluating amateur athletes for progression to an intercollegiate athletic level, there are still many unsolved problems associated with accurately evaluating athletic talent as well as unsolved problems with how best to communicate the performance level of an athlete in a meaningful way that can be favorably received by a university.

Recruiting standards differ among all colleges. These standards are a reflection of the type of university, the level of intercollegiate status, the particular recruiting needs each year to fill roster spots requiring depth and new talent, among many others. Although universities will continue to have their own special recruiting standards or strategies, it is immensely helpful to a university to have detailed reporting on a potential recruit that can be generated by someone outside of the University recruiting staff. The NCAA rules strictly limit the amount of contact a university is able to have with a potential athlete; however, information independently generated by an athlete and sent to university is not subject to many of the rules on direct university contacts. Another problem associated with a University recruiting staff being able to obtain accurate, timely, and reliable data about a recruit is poorly formatted and verified data and descriptions about the athlete.

Prior to an athlete being able to communicate effectively with a recruiting staff, the athlete candidate should have a realistic evaluation of his/her own talents to confirm that a university is a “good fit”; not only in terms of culture, but also a level of intercollegiate performance that matches the athlete. While many athletes may desire to compete at the highest Division I level, there are a very small percentage of athletes who are actually capable of playing at that level. There are still multiple opportunities for scholarships at other levels; and even for those athletes may not require scholarship, having a clear understanding of their talent or performance level is important so the athlete candidate chooses an appropriate level of competition in college.

Considering the above described issues and challenges associated with amateur athletes seeking scholarships, it is one general object of the invention to provide a system and method which generates a standardized output presentation that focuses on relevant, measurable, and reliable data that can be used by an athlete to determine their current athletic capabilities.

It is another object of the invention to provide an automated, computer implemented system and method in which both objective and subjective criteria evaluated may be manipulated in one or more logical expressions to then generate a numerical score that matches the athlete's performance capabilities within a particular collegiate level.

It is yet another object of the invention to provide a system and method in which detailed reporting is generated, specifically tailored for each athlete, in which objective criteria is recorded and conveyed, as well as subjective criteria that is provided in selected evaluation categories.

It is yet another object of the invention to provide a system and method in which automation through computer implementation enables an athlete to adjust performance parameters so the athlete can focus on areas of improvement which could enable the athlete to compete at a higher collegiate level. In this regard, recorded performance can be adjusted in a simulation in which the adjustment provides an updated outcome as to the appropriate collegiate level for the athlete. By adjusting one or more performance criteria, an athlete is given a strategy of how to best focus future time and effort in order to be capable of advancing to the next higher level of collegiate participation.

These objects and others will become apparent from a review the following description of the invention.

### SUMMMARY OF THE INVENTION

The invention is a system and method for evaluating amateur athletes to provide recommendations on placement of the athlete at an appropriate collegiate level. The system and method generally includes analyzing objective data concerning selected measurable parameters relating to selected physical activities relating to the sport of interest. The system and method further generally include observations from an evaluator who observes the performance of an athlete during selected categories relating to the sport of interest.

The invention also includes a computer implemented solution for conveying a recommendation to the athlete regarding the talent or performance grade as compared to a scale that identifies a matching intercollegiate level of competition. The computer implemented solution provides automatic capabilities to convey the recommendation; in one preferred embodiment, the recommendation is conveyed as a numerical score which is then compared to where the numerical score is located within a scale of collegiate levels.

Evaluation of the athlete according to a preferred embodiment of the invention includes evaluating athletic performance according to criteria defined as objective and subjective criteria. These criteria are further divided into selected categories of athletic performance or recorded performance data which could also include physical attributes of the athlete.

For objective criteria, this includes selected criteria of measured athletic performance or parameters in which parameters are selected for measurement which correspond to the particular sport of the athlete.

For subjective criteria, this includes selected criteria of tasks or skills that are required of the athlete in participating in the sport; for example, an evaluation of defensive and offensive skills that can be evaluated by subjecting the athlete to drills or tasks commonly executed within the sport.

For the selected categories for evaluation, point values can be assigned to each category. The athlete is assigned a score after the evaluation for the particular category. The categories are then combined to provide an overall evaluation score, and this evaluation score is then placed on a scale which corresponds to a an intercollegiate level of competition.

According to one particular aspect of the invention, the assignment of point values can be configured within one or more algorithms in which measured/evaluated categories are assigned values and then are used as inputs to the algorithm which then generates the overall score. Data is entered corresponding to the evaluated categories and an overall score may be automatically generated for conveying the current athletic capability in the context of the matching intercollegiate level of competition.

Depending upon the particular sport, there are many different categories that can be used to measure and ultimately convey the current athletic capability of the athlete. The relationship between categories can be weighted to provide a desired emphasis on a particular observed skill or measured parameter. Further, the relationship between categories within an algorithm is not necessarily a simple aggregation of scores for categories that are equally weighted; an algorithm may include one or more specific mathematical functions which are designed to best predict athletic capability of the athlete as compared to collegiate levels of competition.

According to one particular embodiment disclosed herein, the system and method of the invention are described with respect to evaluating an athlete in the sport of baseball or softball. However, it shall be understood that the system and method of the invention are not limited to these sports; the invention is applicable to many other sports including both team sports and individual sports.

According to one preferred embodiment of the invention, a method includes first determining measurable metrics or parameters for a particular sport. These metrics/parameters can be those which are capable of being measured by a device. For example, an electronic timer can be used to record the speed of a measured activity. These metrics/parameters can also be those which are capable of being judged by an evaluator who observes the conducted activity and is able to assign a value to the activity or to otherwise record as to whether the activity was conducted according to a particular standard.

Another step in the method is to determine evaluation categories which can be both objective criteria and subjective criteria. Examples of objective criteria that can be used for baseball/softball include, but are not limited to, foot speed, bat speed, peak hand speed, attack angle of a bat's path, rotational acceleration of a bat, and arm speed of a player throwing a ball. Examples of subjective criteria that can be used for baseball/softball can be drills or activities observed by an evaluator. These drill/activities may include, but are not limited to, hitting drills, live at-bats, outfielder drills, live play as an outfielder, and others.

Another step in the method includes establishing logical category relationships. These category relationships mean how the categories are to be treated within one or more algorithms to determine an overall athletic score for the evaluated athlete. One example of category relationships is assigning each measured metric/parameter an equal weight within an algorithm. Another example of category relationships is assigning different weighted values to selected measured metrics/parameters. Yet another example of category relationships is to only include selected measured metrics/parameters from a group of measured metrics/parameters for inclusion in an algorithm.

Another step in the method is establishing collegiate performance levels. This step includes identifying particular levels within collegiate athletics (such as Division I, II, III, etc.) and determining a range of point values or some other numerical expression that encompasses each particular collegiate athletic level. It should also be understood that there is an overlap between various collegiate levels since it is not possible to exactly define delineations between collegiate levels in terms of measured athletic performance or subjective analysis.

Another step in the method is to confirm and to modify as necessary, the algorithmic applications to be used for the athlete being evaluated. For this step, the method may be simplified to include a predetermined algorithm for the athlete participating in a sport. For example, each particular sport may have a predetermined algorithm based upon a plurality of objective and subjective criteria that are subdivided into the specific categories for each, and the measurements/observations which are then numerical inputs to the algorithm. Alternatively, the algorithms may be modified or adjusted based upon specific attributes of an athlete or based upon a particular sport or level of intercollegiate play being evaluated.

Another step in the method is to gather data about the athlete being evaluated. As mentioned, the data may include measured parameters and an evaluator's observations of skills or tasks performed by the athlete during the evaluation. The data is recorded for each athlete.

Another step in the method is to execute one or more algorithms associated with the evaluation of the athlete in which the data recorded is inputs to the algorithms in order to generate a numerical value which can be concluded as an overall score for the evaluated athlete.

Another step in the method is to generate an output for the user which indicates at least which level of intercollegiate Athletics the athlete is currently at based upon the evaluated performance.

According to one aspect of the invention, the output provided to the user can be regarded as a predictive output in that the overall score of the athlete is an accurate prediction as to which level of collegiate competition the athlete is best suited for success to include optimizing the chances the athlete may be able to obtain a scholarship at that particular level.

Inherent in the method of the invention is the determination of how intercollegiate levels of competition are defined in the context of a point scale. This point scale can be derived from various sources and then combined as an integrated point scale. For example, statistics could be gathered as to how athletes in a particular college level perform for measurable objective criteria. Additional data could be gathered as to how athletes are expected to perform at a particular collegiate level for subjective criteria. A range of scores can then be assigned to a particular collegiate level considering the gathered data.

According to another aspect of the invention, it includes the method of evaluation with a capability for the evaluated athlete to adjust category scores to determine where the athlete could be if the athlete could obtain improved scores in the selected categories. For example, assume an athlete wishes to compete at the Division II level and currently is evaluated at the Division III level for athletic performance capability. The invention includes a mobile application or “app” that allows the athlete to enter new data in the algorithm(s) used in the initial performance evaluation. The algorithm(s) are executed with the new data to produce an updated overall score. The athlete could select from various combinations of evaluated categories with new data to determine various strategies as to how to improve the athlete's overall score making the athlete a better fit for the desired level of competition.

According to one aspect of the invention, it may be considered a method for evaluating an amateur athlete for collegiate placement using a computer to produce user viewable outputs comprising: (1) providing a computer processor, memory, and computer coded instructions associated with evaluation of the amateur athlete comprising objective and subjective criteria, each further subdivided into selected categories; (2) recording a plurality of measurable athletic tasks performed by the athlete as objective criteria data; (3) evaluating other selected categories of athletic tasks performed by the athlete in which evaluation is completed by comments of a designated evaluator; (4) recording the comments of the designated evaluator as subjective criteria data; (5) establishing logical category relationships between said objective and subjective criteria; (6) determining at least one algorithm that sets forth a logical expression incorporating said objective and subjective criteria to generate an overall performance score; (7) automatically generating the overall performance score once the objective and subjective criteria data are entered into the at least one algorithm; and (8) communicating the overall performance score as a function of the performance score placed within a collegiate scoring system comprising a plurality of collegiate levels.

According to another aspect of the invention, it may be considered a system for evaluating an amateur athlete for collegiate placement using a computer to produce user viewable outputs comprising: (1) a computer processor; (2) computer coded instructions executed by said computer processor associated with tasks performed by the amateur athlete of an evaluation of the amateur athlete, comprising objective and subjective criteria, each said criteria further subdivided into selected categories; (3) at least one algorithm executed by said computer coded instructions, said at least one algorithm including at least one logical expression incorporating said objective and subjective criteria; (4) at least one recording device external to said computer processor for recording tasks performed by the amateur athlete during the evaluation, said at least one recording device comprising at least one of a timer, a video camera, an accelerometer, and combinations thereof; (5) a memory coupled to said computer processor for storing data corresponding to the performed tasks; (6) an output associated with said computer processor and said at least one algorithm that displays at least a numerical score corresponding to an evaluated overall performance of said amateur athlete; and (7) a scale associated with levels of collegiate athletics, said scale enumerating said levels of collegiate athletics within numerical score ranges; and wherein said output further includes an indication of the numerical score as a function of said scale thereby providing the amateur athlete an indication as to his/her current level of performance for competing at a matching collegiate level of competition.

The system of the invention adopts the functionality described above with respect to the method of the invention and further includes at least a general-purpose computer and one or more measurement devices that are used to measure the selected metrics/parameters. The general-purpose computer at least includes a processor, memory, and a communications capability to generate outputs to convey information to the evaluated athlete. The conveying information may include a printed report, a screen display, and others. The system may further include a communications capability for one or more mobile devices which communicate directly with the computer to transmit recorded data and recorded evaluator comments about an athlete's performance during an evaluation. Also according to the system, some automation is adopted in which an overall score is automatically generated when sufficient data is received to satisfy variables in one or more algorithms which are used to generate the overall score. Accordingly, user manipulation is minimized once sufficient data has been entered.

In connection with a computer of the invention, it may be further defined to include any general purpose personal computers (including, merely by way of example, personal computers and/or laptop computers running various versions of Microsoft's Windows® and/or Apple® operating systems) and/or workstation computers running any of a variety of commercially-available LINUX®, UNIX® or LINUX®-like operating systems. These computers may also have any of a variety of applications, including for example, database client and/or server applications, and web browser applications. Alternatively, the computers may be any other electronic device, such as a thin-client computer, Internet-enabled mobile telephone, and/or personal digital assistant, capable of communicating via a network and/or displaying and navigating web pages or other types of electronic documents.

In connection with sharing or distributing outputs of the system and method of the invention, the invention may further include a communications network that can support data communications using any of a variety of commercially-available protocols, including without limitation TCP/IP, SNA, IPX, AppleTalk®, and the like. Merely by way of example, the communications network maybe a local area network (“LAN”), such as an Ethernet network, a Token-Ring network and/or the like; a wide-area network; a virtual network, including without limitation a virtual private network (“VPN”); the Internet; an intranet; an extranet; a public switched telephone network (“PSTN”); an infra-red network; a wireless network (e.g., a network operating under any of the IEEE 802.11 suite of protocols, the BluetoothTM protocol known in the art, and/or any other wireless protocol); and/or any combination of these and/or other networks.

The communication network may also include one or more server computers. One type of server may include a web server used to process requests for web pages or other electronic documents from user computers. The web server can be running an operating system including any of those discussed above, as well as any commercially-available server operating systems. The web server can also run a variety of server applications, including HTTP servers, FTP servers, CGI servers, database servers, Java servers, and the like. In some instances, the web server may publish operations available as one or more web services.

The communication network may further include one or more file and/or application servers, which can, in addition to an operating system, include one or more applications accessible by a client running on one or more of the user computers and network. The file/application server(s) may be one or more general purpose computers capable of executing programs or scripts in response to the user computers and network. As one example, the server may execute one or more web applications. The web application may be implemented as one or more scripts or programs written in any programming language, such as Java®, C, C#™ or C++, and/or any scripting language, such as Perl, Python, or TCL, as well as combinations of any programming/scripting languages. The application server(s) may also include database servers, including without limitation those commercially available from Oracle®, Microsoft, Sybase®, IBM® and the like, which can process requests from database clients running on a user computer.

An application server may create web pages dynamically for displaying the functionality associated with the system to include user interfaces that include specific information about the tasks performed by the athlete during evaluation. The web pages created by the web application server may be forwarded to a user computer via a web server. Similarly, the web server may be able to receive web page requests, web services invocations, and/or input data from a user computer and can forward the web page requests and/or input data to the web application server.

In connection with memory of a computer of the invention, it may include one or more databases that can reside in a variety of locations. By way of example, a database may reside on a storage medium local to (and/or resident in) one or more of the computers. Alternatively, it may be remote from any or all of the computers and in communication with one or more of these computers by the communication network.

The system of the invention may further include one or more mobile devices such as “smart phones”. These mobile devices communicate with the communication network as by a web interface. The communication network may also represent a cloud provider who facilitates communication with communication endpoints or computers of a customer network. The mobile devices may communicate with any other of the computers in the system through the communication network The mobile devices have their own internal computer processing capabilities with integral computer processors and other supporting hardware and software. The mobile devices may be specially configured to run a mobile software application(s) in order to view user interfaces and to view and update system data. All of the functionality associated with the system as applied to the computers may be incorporated in the mobile devices as modified by mobile software applications especially adapted for the mobile device hardware and operating systems. In connection with operating systems, it should therefore be understood that the mobile devices are not limited to any particular operating system, Apple iOS and Android-based systems being two examples.

In accordance with any computer of the invention, these may be further described as general-purpose computers with elements that cooperate to achieve multiple functions normally associated with general purpose computers. For example, the hardware elements may include one or more central processing units (CPUs) for processing data. The computers may further include one or more input devices (e.g., a mouse, a keyboard, etc.); and one or more output devices (e.g., a display device, a printer, etc.). The computers may also include one or more storage devices. By way of example, storage device(s) may be disk drives, optical storage devices, solid-state storage device such as a random access memory (“RAM”) and/or a read-only memory (“ROM”), which can be programmable, flash-updateable and/or the like.

Any computer and server described herein may include a computer-readable storage media reader; a communications peripheral (e.g., a modem, a network card (wireless or wired), an infra-red communication device, etc.); working memory, which may include RAM and ROM devices as described above. The server may also include a processing acceleration unit, which can include a DSP, a special-purpose processor and/or the like.

The computer-readable storage media reader can further be connected to a computer-readable storage medium, together (and, optionally, in combination with storage device(s)) comprehensively representing remote, local, fixed, and/or removable storage devices plus storage media for temporarily and/or more permanently containing computer-readable information. The computers and serve permit data to be exchanged with the network 110 and/or any other computer, server, or mobile device.

The computers and server also comprise various software elements and an operating system and/or other programmable code such as program code implementing a web service connector or components of a web service connector. It should be appreciated that alternate embodiments of a computer may have numerous variations from that described above. For example, customized hardware might also be used and/or particular elements might be implemented in hardware, software (including portable software, such as applets), or both. Further, connection to other computing devices such as network input/output devices may be employed.

It should also be appreciated that the method described herein may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, ROMs, RAMs, EPROMs, EEPROMs, magnetic or optical cards, flash memory, or other types of machine-readable mediums suitable for storing electronic instructions. Alternatively, the methods may be performed by a combination of hardware and software.

The term “computer coded instructions” as used herein shall be broadly interpreted to include all information processed by a computer processor, a microcontroller, or processed by related computer executed programs communicating with the computer coded instructions. The computer coded instructions may also be referred to herein as software. Software therefore includes computer programs, libraries, and related non-executable data, such as online documentation or digital media. Executable code makes up definable parts of the software and is embodied in machine language instructions readable by a corresponding data processor such as a central processing unit of the computer. The software may be written in any known programming language in which a selected programming language is translated to machine language by a compile, interpreter or assembler element of the associated computer.

Considering the foregoing, it is an overall objective of the invention to provide a predictive output for an evaluated athlete that places the athlete at the best fit in terms of where the athlete's stands at the evaluated capabilities and a match for the level of collegiate competition.

Although the invention is especially adapted for providing outputs to an evaluated athlete, the invention may also be directed for transfer of information to recruiting departments of colleges seeking recruits. Considering the vast amount of information that a college receives in any particular recruiting season, the outputs of the invention provide a concise, accurate, and standardized format that can be used by recruiting departments.

Other features and advantages of the invention will become apparent from a review of the following detailed description taken in conjunction with the figures and claims.

### BRIEF DESCRIPTION OF THE DRAWINGS

FIG. 1 is a simplified flow diagram of a method of the invention;

FIG. 2 is an output of the invention showing evaluated category scores as compared with a collegiate scale as well as an overall evaluated score compared with a collegiate scale;

FIG. 3 is an output of the invention illustrating evaluator notes regarding offensive performance of the evaluated athlete within a particular sport;

FIG. 4 is another output of the invention illustrating evaluator notes regarding offensive performance of the evaluated athlete;

FIG. 5 is another output of the invention illustrating evaluator notes regarding defensive performance of the evaluating athlete within the particular sport;

FIG. 6 is another output of the invention illustrating evaluator notes regarding next steps that the athlete may take to improve performance, subdivided in comments regarding athletic skills, physical training, and mental or attitude training; and

FIG. 7 is a simplified flow diagram of another method of the invention corresponding to a method for an evaluated athlete to adjust category scores and/or components of evaluated categories to determine if the athlete can be reclassified at a different collegiate level, the method being especially adapted for implementation on a computer or mobile device with a corresponding software application.

### DETAILED DESCRIPTION

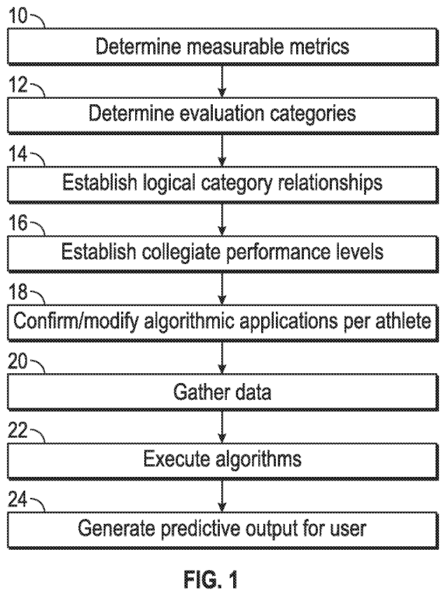

FIG. 1 illustrates a simplified flow diagram of a method of the invention according to one preferred embodiment. Although the method is presented in a particular order, it should be understood that the method does not have to be executed in the same order. One step 10 in the method is to determine measurable metrics. The term “metrics” means a type of measurable activity, such as those that are capable of being measured by a device, but also those that are capable of being judged according to criteria or standards, such as those which are capable of being judged by an evaluator who observes the conducted activity. The evaluator who judges an activity can assign a basis for success or failure or may otherwise rank or score the observed activity. Further, the evaluator may provide comments which form a subjective aspect of the evaluated performance of the athlete. Examples of measuring devices include, but are not limited to, a timer, a video camera, an accelerometer, and combinations thereof.

Another step in the method is determining evaluation categories 12 that can be objective, subjective, or combinations thereof. In connection with a preferred embodiment of the invention disclosed herein, baseball/softball is the example sport for which both subjective and objective criteria are explained.

Another step in the method includes establishing the logical category relationships 14 so that measured activities can be considered as a whole in forming a conclusion as to the level of performance that the evaluated athlete is currently at in comparison to a collegiate level of performance. The logical relationships form the components or elements of one or more algorithms to determine an overall athletic score for the evaluated athlete. Examples of category relationships is assigning each measured metric an equal weight within the algorithm; assigning different weighted values to selected measured metrics; and selectively choosing which measured metrics to use within an algorithm.

Another step in the method is to establish collegiate performance levels 16. The performance levels can be defined by a point scale in which each college division or level falls in a range of point values. A point-based scoring system can also be applied to each measured activity with respect to a corresponding collegiate performance levels, thereby providing athlete an indication of where their performance falls for not only an overall performance, but for each measured activity.

Yet another step in the method is to confirm and modify, as necessary, algorithmic applications per athlete 18. Algorithms may be developed for each sport and may be further modified taking into account special circumstances of the athlete looking to be rated at a particular level for a particular university or group of universities. For example, a high performing high school athlete who wishes to attend a Division I school may be provided a different algorithmic function as compared to another high school athlete who simply wishes to be considered at a level for Division III or NAIA participation. Certain evaluated categories may be weighted differently within the algorithms as a reflection of the nature of the collegiate levels and other factors. Therefore, it should be understood that the algorithmic functions of the invention can be tailored on a wide-ranging basis to match the specific information that the athlete is seeking in terms of qualifying for a particular collegiate level.

Another step in the method is to gather the data 20. This step involves adding the athlete participate in an evaluation in which selected objective in subjective criteria are evaluated. The evaluation can be conducted over a period of time, or may be conducted within a single evaluation event. For objective criteria, devices such as timers may communicate remotely with a computer for transfer and storage of data. Alternatively, data can be uploaded to a computer manually. It is contemplated within the scope of the invention that the recording and transfer of data to a computer can be conducted in many different ways to include use of wireless data transfer by use of mobile communication devices operated by the evaluators.

Another step in the method is to execute the algorithms 22 so that the athlete can be numerically scored or otherwise ranked as to a performance level that can then be compared to a collegiate performance level.

A final step in the method is then to generate a predictive output for the user 24 which may include various forms such as a printed report or user screens on a software application.

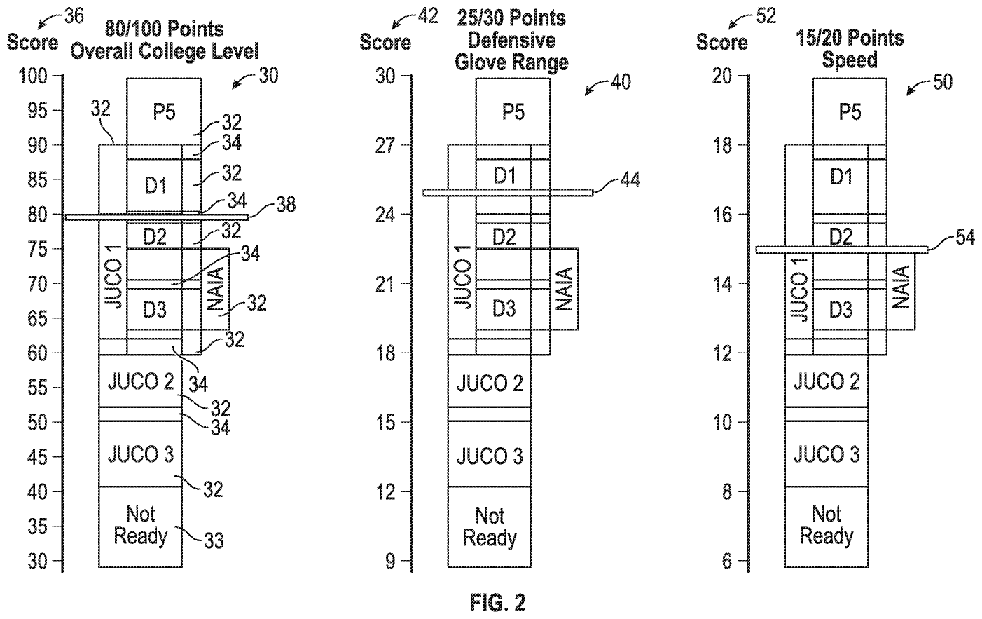

Referring to FIG. 2, this figure is an exemplary output provided to an evaluated athlete showing evaluated category scores as compared with a collegiate scale as well as overall evaluated score compared with an overall collegiate scale. More specifically, FIG. 2 illustrates an overall college-level scale 30 with scores 36 which match a level of collegiate performance. Various collegiate participation levels 32 are shown to include Division I (D1), Division II (D2), Division III (D3), NAIA, Junior College 1 (JUCO 1), Junior College 2 (JUCO 2) and Junior College 3 (JUCO 3). The highest level is shown as a Power 5 University (P5) which corresponds to those universities that are the most competitive at the collegiate level for the particular sport. In the particular example of FIG. 2, the evaluated athlete obtained a score of 80 out of a possible 100 points.

The collegiate level scale also shows a range of scores indicating the evaluated athlete is not ready for any collegiate level (NOT READY). College eligible scores are shown as ranging between point values of 40 to 100. However, it should be understood that this scoring range is exemplary, and scoring can be modified to adopt other numerical ranges.

FIG. 2 also shows point ranges for each of the levels of collegiate participation. It is observed that the collegiate levels overlap one another, illustrated as overlapping areas 34. For example, JUCO 1 represents that there are some junior-college programs that compete at very high levels and athletes attending those junior colleges may have performance abilities that match D1 athletes. The range for JUCO 1 is particularly large, noting that the JUCO 1 level of college performance expands the range of D1-D3 as well as NAIA. Performance line 38 is the indicator of the points awarded to the evaluated athlete. In FIG. 2, the performance line intersects a number of different collegiate levels, namely, JUCO 1, D1 and D2. This performance line 38 can be more specifically described as indicating that the evaluated athlete has an overall score that places the athlete at a performance or talent level which is at the lower end of a D1 school, the upper end of a D2 school, and at the mid or upper range of a JUCO 1 school. One can appreciate that depending upon where the performance line is located, the evaluated athlete is provided a host of options in terms of what schools the athlete may best be qualified for.

FIG. 2 also shows scoring for particular evaluated categories, namely, defensive skills 40, hitting 60, speed 50, and arm velocity 70. The corresponding scoring scales for these evaluated categories are shown as scoring scales 42, 52, 62, and 72. Similar to the overall college-level scale, the other evaluated categories also include a college-level scale for each category along with a performance line indicating the performance of the evaluated athlete. For defensive skills 40, the evaluated athlete has a performance line 44 placing the athlete at mid-range for D1 and the upper end of JUCO 1. For speed 50, the performance line 54 is located mid-range for D2, also intersecting the upper end of NAIA and mid-range for JUCO 1. For hitting 60, the performance line 64 is located mid-range D2 and also intersecting midrange JUCO 1. For the arm velocity category 70, the performance line is located mid-range for D1 and also intersecting the upper end of JUCO 1. As also shown, the evaluated athlete obtained a score of 25 out of 30 points for the defensive skills 40, a score of 15 out of 20 for speed 50, a score of 23 out of 30 for hitting 60, and a score of 17 out of 20 for arm velocity 70.

By providing an athlete these performance lines as an indication of their performance or talent level in comparison to various collegiate levels, to include not only an overall performance line but also detailed performance lines for specific categories, the athlete is able to focus on those specific areas that may require improvement in order to achieve a higher level of potential collegiate eligibility.

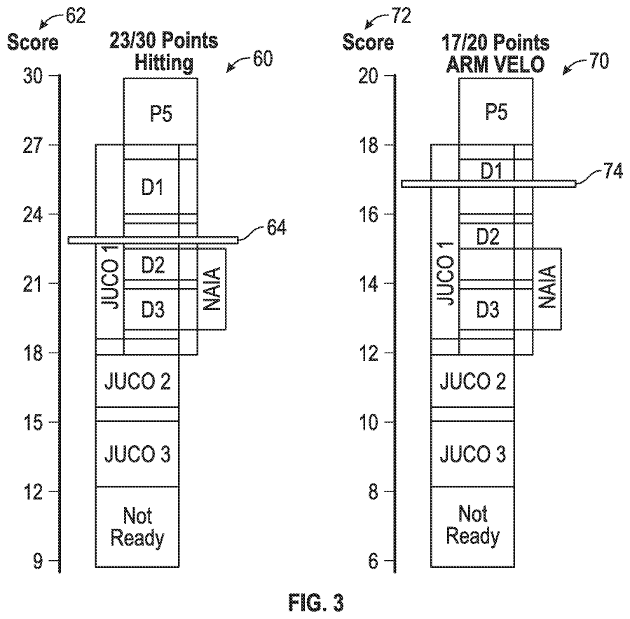

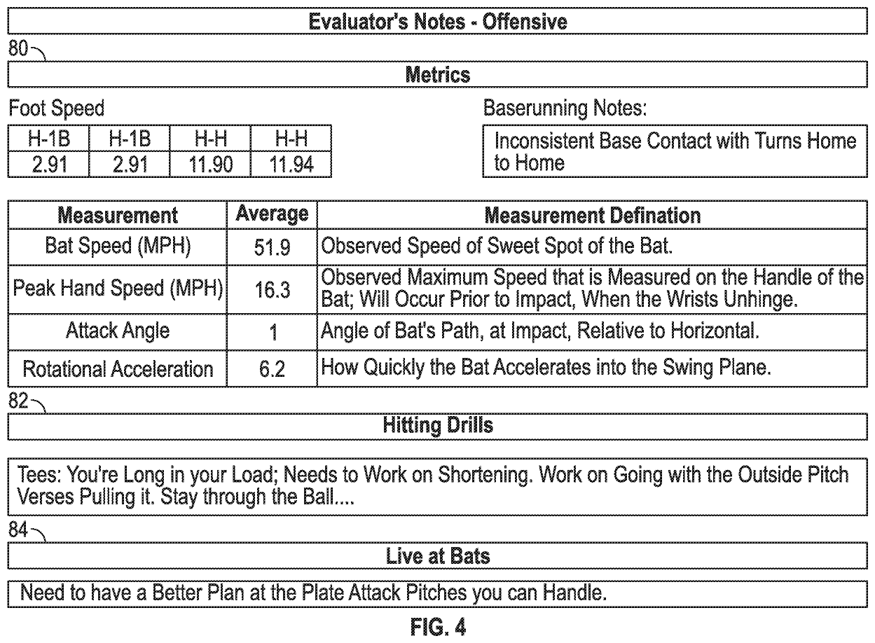

Referring to FIGS. 3 and 4, additional outputs are provided to the athlete, namely, offensive notes from the evaluator and measured metrics 80. These measured metrics are simply an example of the type of objective criteria that can be recorded corresponding to objective categories as one or more variables in an algorithm(s) determining the points to be awarded to the evaluated athlete. The specific metrics illustrated are those that are attributed to Blastmotion®, a company in the US who provides solutions for improving athletic performance. Also shown is a hitting drills category 82 and comments provided by the evaluator. Although not shown, the evaluator may be given the option to also rate the evaluated athlete for competency in hitting drills, and the evaluator could assign a point value indicating the level of success or deficiency of the athlete in executing the hitting drills. This figure also illustrates yet another category, namely, live at bats 84. Again, comments are provided by the evaluator and the evaluator may be given the option to rate the athlete for competency in executing the live at bats.

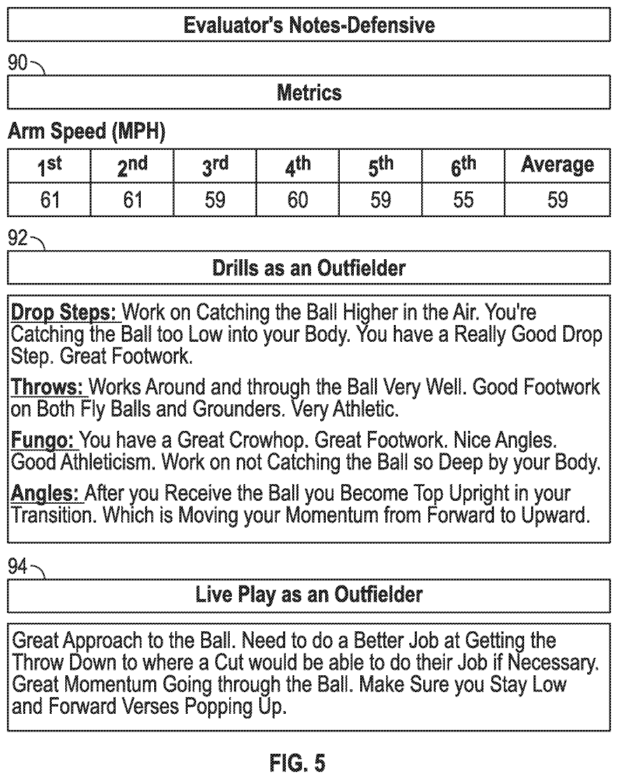

Referring to FIG. 5, yet another output is provided to the athlete, namely, defensive notes from the evaluator to include measured defensive metrics 90. The particular defensive metric shown is arm speed evaluated over six throwing tries. This figure also illustrates two additional evaluator notes; outfielder drills 92 and outfielder live play 94. As with the description set forth in FIG. 3, the evaluator may be given the option to rate the athlete for competency in executing the categories which can then form a part of variables as inputs to an algorithm.

It should be understood that regardless of whether the evaluated categories for evaluator notes become variables or inputs for an evaluation algorithm, the evaluator notes as subjective information given to the athlete are very valuable. Although sports are increasingly evaluated analytically, the coaching aspect of a sport cannot be underestimated in terms of importance. A skilled evaluator may be able to provide the subject comments which give the athlete a fairly comprehensive plan as to how improvement can be approached.

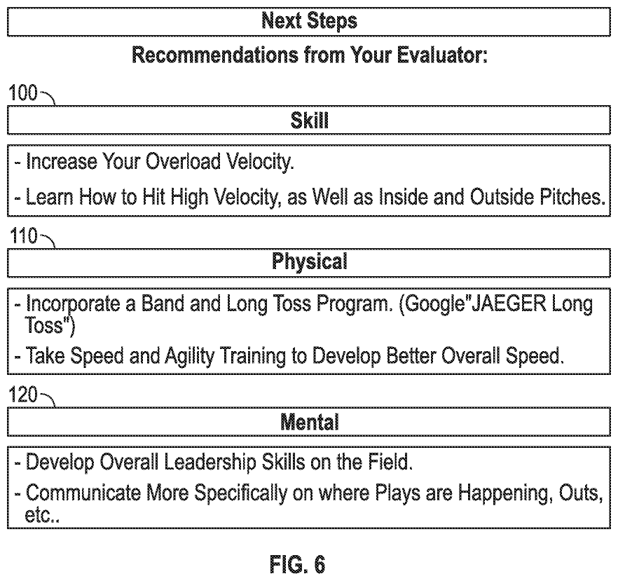

FIG. 6 is yet another system output, namely, recommended next steps from the evaluator. These recommendations are provided in three categories, namely, skill 100, physical 110, and mental 120. These recommendations can be regarded as a general summary of the evaluated categories in which the evaluated are can summarize the important points for the athlete to consider in the future.

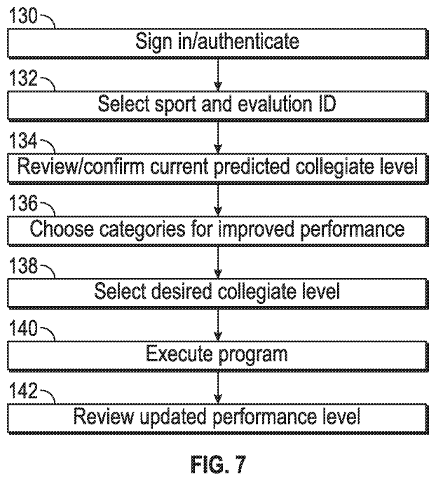

FIG. 7 is a simplified schematic diagram illustrating another method of the invention, namely, a method for an evaluated athlete to adjust category scores and/or components of evaluated categories to determine if the athlete can be reclassified at a different collegiate level, the method being especially adapted for implementation on a computer or mobile device with a corresponding software application. Referring to the figure, a first step 130 may include the athlete signing in and authenticating his/her identity as one who has participated in the performance evaluation. At step 132, a menu can be provided on the app enabling selection of the sport that was evaluated and an evaluation ID, such as a particular identifying number associated with the athlete's evaluation. At step 134, the athlete reviews and confirms the current predicted collegiate level, that is, where the performance line indicated that the athlete was a best fit. At step 136, the athlete chooses evaluation categories for improved performance. For example, if the athlete performed particularly poor in one category during the evaluation, the athlete may wish to select this category as one to be improved and to enter an improved score to be obtained for that category. In other words, the athlete selects an improved score as compared to the actual evaluated score. At step 138, the athlete is provided an option as to which collegiate level the athlete was seeking to qualify for. At step 140, the algorithm/program is executed with the substituted score. At step 142, an updated prediction or updated performance level is provided to the user, such as a performance line that has now shifted to a higher level. The athlete could manipulate one or more category scores in order to determine strategies for improved performance that might better qualify the athlete for a particular collegiate level.

While the invention has been described with respect to various preferred embodiments, it should be understood that the invention is not strictly limited to such embodiments, and the scope of the invention should be considered commensurate with the scope of the claims appended hereto.

What is claimed is:

1. A method for evaluating an amateur athlete for collegiate placement comprising:
providing a computer processor, memory, and computer coded instructions associated with evaluation of the amateur athlete comprising objective and subjective criteria, each further subdivided into selected categories;
recording a plurality of measurable athletic tasks performed by the athlete as objective criteria data;
evaluating other selected categories of athletic tasks performed by the athlete in which evaluation is completed by comments of a designated evaluator;
recording the comments of the designated evaluator as subjective criteria data;
establishing logical category relationships between said objective and subjective criteria;
determining at least one algorithm that sets forth a logical expression incorporating said objective and subjective criteria to generate an overall performance score;
automatically generating the overall performance score once the objective and subjective criteria data are entered into the at least one algorithm; and
communicating the overall performance score as a function of the performance score placed within a collegiate scoring system comprising a plurality of collegiate levels.

2. A system for evaluating an amateur athlete for collegiate placement comprising:
a computer processor;
computer coded instructions executed by said computer processor associated with tasks performed by the amateur athlete associated of an evaluation of the amateur athlete, comprising objective and subjective criteria, each said criteria further subdivided into selected categories;
at least one algorithm executed by said computer coded instructions, said at least one algorithm including at least one logical expression incorporating said objective and subjective criteria;
at least one recording device external to said computer processor for recording tasks performed by the amateur athlete during the evaluation, said at least one recording device comprising at least one of a timer, a video camera, an accelerometer, and combinations thereof;
a memory coupled to said computer processor for storing data corresponding to the performed tasks;
an output associated with said computer processor and said at least one algorithm that displays at least a numerical score corresponding to an evaluated overall performance of said amateur athlete; and
a scale associated with levels of collegiate athletics, said scale enumerating said levels of collegiate athletics within numerical score ranges; and wherein said output further includes an indication of the numerical score as a function of said scale thereby providing the amateur athlete an indication as to his/her current level of performance for competing at a matching collegiate level of competition.

3. A method for evaluating an amateur athlete for collegiate placement comprising:
determine measurable metrics;
determine evaluation categories
establish logical category relationships;
establish collegiate performance levels;
create at least one at least one algorithm including at least one logical expression incorporating objective and subjective criteria associated with an evaluation of the amateur athlete;
confirm and modify algorithmic applications per athlete;
gather data and store in a computer that processes the at least one algorithm;
execute the at least one algorithm in the computer; and
generate a predictive output for a user, wherein said predictive output includes a numerical score of an evaluated amateur athlete as a function of scale enumerating levels of collegiate athletics within numerical score ranges, said scale thereby providing the amateur athlete an indication as to his/her current level of performance for competing at a matching collegiate level of competition.
